(19) United States
(12) Patent Application Publication
(10) Pub. No.: US 2017/0325851 A1
(43) Pub. Date: Nov. 16, 2017

(54) APPARATUS FOR PREVENTING VERTEBRA DISPLACEMENT AFTER SPINAL FUSION SURGERY

(71) Applicant: Frank Acosta, Los Angeles, CA (US)

(72) Inventor: Frank Acosta, Los Angeles, CA (US)

(21) Appl. No.: 15/153,557

(22) Filed: May 12, 2016

(51) Int. Cl. A61B 17/70; A61B 17/00

(52) U.S. Cl. CPC A61B 17/7053; A61B 17/707; A61B 2017/00969; A61B 2017/00004

(57) ABSTRACT

The present disclosure includes systems, apparatuses, and devices for impeding vertebra displacement. In accordance with embodiments of the present disclosure, a system for impeding vertebra displacement includes an elongated member. The elongated member includes a proximal end and a distal end. The system also includes a first fastener attached to the proximal end and configured to be coupled to a first vertebra. Additionally, the system includes a second fastener attached to the distal end and configured to be coupled to a second vertebra. The system further includes an implant coupled between the second vertebra and a third vertebra and configured to promote a fusion of the second vertebra and the third vertebra.

### TECHNICAL FIELD

The present disclosure relates generally to spinal fusion surgery and associated treatments and techniques. More particularly, the present disclosure is directed to apparatuses, systems, methods, and devices, for impeding and/or preventing vertebra displacement after spinal fusion surgery.

### BACKGROUND

As depicted in FIG. 1A, the typical human spine (vertebral column 100) typically includes twenty-four articulating vertebrae and nine fused vertebrae. The thirty-three total vertebrae in vertebral column 100 are split into five regions: cervical, thoracic, lumbar, sacral, and coccygeal. Each vertebrae is separated by intervertebral discs, which act as ligaments to hold the vertebrae together. Generally, the structure of a typical vertebra includes a main vertebral body and a vertebral arch, which further includes pair of pedicles and a pair of laminae, enclosing a vertebral foramen and supporting seven processes—four articular (two each of a superior and an inferior process), two transverse, and one spinous.

The main vertebral body is connected to the two pedicles, which are directed toward the posterior. The pedicles are each connected to the laminae, and from each of these junctions the superior articular processes and the inferior articular processes project upward and downward, respectively. The spinous process is directed obliquely downward, and extends from the junction of the two laminae. The transverse processes project from where the lamina joins the pedicle, between the superior and inferior articular processes. The body, pedicles, and laminae define a vertebral foramen. The vertebral foramina are formed when the vertebrae are articulated to each other, and extend from the first cervical vertebrae to the last lumbar vertebrae, housing the spinal cord and associated meninges. The cervical, thoracic, and lumbar regions are discussed briefly below.

The cervical region of vertebral column 100 typically consists of seven vertebral bones that allow for movement of the neck and head. Cervical vertebrae (C1-C7) include a small body, pedicles directed laterally and toward the posterior, laminae, the articular processes (superior and inferior), and the transverse processes. Cervical vertebrae (C1-C7) are characterized by their smaller size, and are usually easily distinguished by the presence of a foramen in each transverse process.

The thoracic region of vertebral column 100 typically consists of twelve vertebral bones, with transverse processes that have surfaces that articulate with the ribs. The thoracic vertebrae (Th1-Th12) are usually distinguished by the facets present on the vertebral bodies that allow for articulation with the heads of the ribs, and the facets on the transverse processes of the first ten thoracic vertebrae that allow for articulation with the tubercles of the ribs. There is little normal motion of the vertebrae in the thoracic region, in comparison to the cervical and lumbar regions.

The lumbar region of vertebral column 100 typically consists of five vertebral bones (L1-L5). These vertebrae are typically the largest moveable segments of vertebral column 100, supporting more weight than any other vertebrae. Structurally, the vertebral body of each lumbar vertebra (L1-L5) is relatively large, with strong pedicles, broad and short laminae, and a thick, broad spinous process.

Several spinal disorders may affect the curvature, motion, and/or functionality of vertebral column 100. For instance, degenerative disc disease, spinal disc herniation, fractures, tumors, or scoliosis all affect vertebral column 100 and can result in severe pain or neurological deficits. Also, spinal injury may affect the vertebral column 100, and may require correction.

Correction of the spinal disorders mentioned above can be achieved via a spinal fusion procedure, a surgical technique that joins or fuses two or more vertebrae. Also, correcting the spinal curvature can result in relief of pain caused by abnormal motion of the vertebrae. One method of spinal fusion involves fusing the affected vertebrae using bone tissue grafts harvested from the patient or a donor, and utilizes the patient's natural bone growth processes to fuse the vertebrae. Another method of spinal fusion involves implanting an instrumentation into and/or onto the vertebrae to support correction of spinal curvature, where such instrumentation effectively fuses the vertebrae. The implanted instrumentation may also encourage natural bone growth between the vertebrae.

Typical spinal fusion instrumentation includes pedicle screws affixed to a support rod, as shown in FIG. 1B. FIG. 1B illustrates a diagram depicting a close-up side view of vertebral column 102. As shown in FIG. 1B, the vertebral column includes vertebrae 110, 115, 120, 125, and 130. Also shown in FIG. 1B is instrumentation 105, which includes support rod 135 and fixture 145. The surgical procedure used in spinal fusion generally involves implanting pedicle screws, such as pedicle screw 155, into the pedicles on one side of two adjacent vertebrae. Support rod 135 is then affixed to the protruding head portion of each respective pedicle screw 155 by fixture 145. The same process is applied to the other side of the vertebrae of vertebral column 102.

One problem with the above described spinal fusion procedure and instrumentation implantation is that, following the procedure, one or more vertebrae near the instrumentation may become displaced (e.g., laterally). For example, the vertebra located vertically adjacent to the fused vertebrae, such as one or more of vertebrae 115, 120, 125, and 130, may displace laterally from the desired or natural curvature and alignment of vertebral column 102.

### BRIEF SUMMARY OF THE DISCLOSURE

In view of the above shortcomings in conventional spinal fusion techniques and instrumentation, there exists a need for providing additional support to the spine following spinal fusion surgery. In particular, there exists a need for providing such support to vertebrae near, including vertically adjacent to, fused vertebrae joined together with instrumentation and/or bone growths. In this connection, embodiments of the present disclosure include apparatuses, systems, methods, and devices, capable of impeding and/or preventing vertebra displacement after spinal fusion surgery.

Aspects of the present disclosure involve an apparatus for impeding vertebra displacement after a spinal fusion surgery. The apparatus includes an elongated tissue having a first end and a second end. The elongated tissue may be a cadaveric tissue harvested from a human cadaver. The apparatus includes a first fastener attached to the first end and configured to be coupled to a first vertebra. Further, the apparatus includes a second fastener attached to the second end and configured to be coupled to a second vertebra coupled to a third vertebra via a spinal fusion instrumentation. The first fastener may be wrapped around the first end. The second fastener may be wrapped around the second end. The first fastener may pass through the first end. The second fastener may pass through the second end. The first fastener may include a first strand. The second fastener may include a second strand. One or more of the first and second strands may include one or more of thread-like material, wire, and flexible synthetic material. The first strand may include a first loop attachable to the first vertebra. The second strand may include a second loop attachable to the second vertebra. The first fastener may be configured to be coupled to the first vertebra via an optional first anchor feature. The second fastener may be configured to be coupled to the second vertebra via an optional second anchor feature. If present, the first anchor feature may include a first passage formed in the first vertebra. If present, the second anchor feature may include a second passage formed in the second vertebra. If present, the first anchor feature may include a first fixation protrusion coupled to the first vertebra. If present, the second anchor feature may include a second fixation protrusion coupled to the second vertebra.

Additional aspects of the present disclosure involve an apparatus. The apparatus includes first and second fasteners attachable to vertically adjacent vertebrae. At least one of the vertically adjacent vertebrae is coupled to an other vertebra by way of a spinal fusion. The apparatus further includes a tissue that includes a first end coupled to the first fastener. The tissue also includes a second end coupled to the second fastener. The tissue may be sized in length to span a distance between the vertically adjacent vertebrae when the first fastener is attached to a first of the vertically adjacent vertebrae and the second fastener is attached to a second of the vertically adjacent vertebrae. The tissue may be selected so as to impede lateral displacement of the first of the vertically adjacent vertebrae relative to the second of the vertically adjacent vertebrae after a spinal fusion surgery. In embodiments, the spinal fusion includes one or more of a bone graft, a spinal fusion instrumentation, and a bone growth.

In example implementations of the apparatus, the first fastener includes a first strand having a first loop attachable to a first protrusion that protrudes from a first of the vertically adjacent vertebrae. In example implementations, the second fastener includes a second strand having a second loop attachable to a second protrusion that protrudes from a second of the vertically adjacent vertebrae. In some such cases, the spinal fusion includes a spinal fusion instrumentation. In such cases, one or more of the first and second fastener is attachable to the spinal fusion instrumentation.

Yet additional aspects of the present disclosure involve a system for impeding vertebra displacement. The system includes an elongated member that includes a proximal end and a distal end. The system also includes a first fastener attached to the proximal end and configured to be coupled to a first vertebra. Further, the system includes a second fastener attached to the distal end and configured to be coupled to a second vertebra. And the system includes an implant coupled between the second vertebra and a third vertebra and configured to promote a fusion of the second vertebra and the third vertebra.

In example deployments of the system, the first and second vertebrae and the second and third vertebrae are vertically adjacent. In some such examples, the elongated member is sized so as to span a distance between the first and second vertebrae when the first fastener is coupled to the first vertebra and the second fastener is coupled to the second vertebra. In example deployments, the implant comprises a spinal fusion instrumentation. In some such deployments, the second fastener is configured to be coupled to the second vertebra by attaching to the spinal fusion instrumentation. In some such deployments, the first fastener is configured to be coupled to the first vertebra by attaching to an anchor feature coupled to the first vertebra. In some example deployments, the first fastener includes a first loop attachable to the first vertebra. In some such deployments, at least a portion of the first fastener is wrapped around the proximal end of the elongated member. In some example deployments, the second fastener includes a second loop attachable to the second vertebra. In some such deployments, at least a portion of the second fastener is wrapped around the distal end.

### BRIEF DESCRIPTION OF THE DRAWINGS

Further aspects of the present disclosure will be more readily appreciated upon review of the detailed description of the various disclosed embodiments, described below, when taken in conjunction with the accompanying figures.

FIG. 1A is a diagram depicting a side view of a human spine or vertebral column that may be discussed in connection with embodiments of the disclosure.

FIG. 1B is a diagram depicting a close-up side view of a human spine or vertebral column that may be discussed in connection with embodiments of the disclosure, including an example spinal fusion device implanted on a spinal section of the vertebral column during spinal fusion surgery, in accordance with embodiments of the disclosure.

FIG. 1C is a diagram depicting a close-up side view of an example system for impeding vertebra displacement, including an example spinal fusion device implanted on a spinal section of a vertebral column during spinal fusion surgery, as well as an example apparatus for impeding vertebra displacement, in accordance with embodiments of the disclosure.

FIG. 2 is a diagram depicting a posterior view of an example system for impeding vertebra displacement, including example spinal fusion devices implanted on a spinal section of a vertebral column during spinal fusion surgery, as well as example apparatuses for impeding vertebra displacement, in accordance with embodiments of the disclosure.

FIG. 3 is a diagram depicting a posterior view of an example system for impeding vertebra displacement, including bone growths on a spinal section of a vertebral column following spinal fusion surgery, as well as example apparatuses for impeding vertebra displacement, in accordance with embodiments of the disclosure.

FIG. 4 is a diagram depicting an example apparatus for impeding vertebra displacement, in accordance with embodiments of the disclosure.

FIG. 5 is a diagram depicting an example apparatus for impeding vertebra displacement, in accordance with embodiments of the disclosure.

FIG. 6A is a diagram depicting an example of a vertebral column that includes vertebrae configured for attachment of an apparatus for impeding vertebra displacement, in accordance with embodiments of the disclosure.

FIG. 6B is a diagram depicting an example of a vertebral column that includes vertebrae configured for attachment of an apparatus for impeding vertebra displacement, in accordance with embodiments of the disclosure.

FIG. 6C is a diagram depicting an example of a vertebral column that includes vertebrae with an example apparatus for impeding vertebra displacement attached thereto, in accordance with embodiments of the disclosure.

FIG. 6D is a diagram depicting an example of a vertebral column that includes vertebrae with an example apparatus for impeding vertebra displacement attached thereto, in accordance with embodiments of the disclosure.

FIG. 6E is a diagram depicting an example of a vertebral column that includes vertebrae with an example apparatus for impeding vertebra displacement attached thereto, in accordance with embodiments of the disclosure.

FIG. 6F is a diagram depicting an example of a vertebral column that includes vertebrae with an example apparatus for impeding vertebra displacement attached thereto, in accordance with embodiments of the disclosure.

FIG. 7 is an operational flow diagram illustrating various operations that may be performed in accordance with embodiments of the disclosure.

FIG. 8 is an operational flow diagram illustrating various operations that may be performed in accordance with embodiments of the disclosure.

FIG. 9 is an operational flow diagram illustrating various operations that may be performed in accordance with embodiments of the disclosure.

FIG. 10 is an operational flow diagram illustrating various operations that may be performed in accordance with embodiments of the disclosure.

The figures are described in greater detail in the description and examples below, are provided for purposes of illustration only, and merely depict typical or example embodiments of the disclosure. The figures are not intended to be exhaustive or to limit the disclosure to the precise form disclosed. It should also be understood that the disclosure may be practiced with modification or alteration, and that the disclosure may be limited only by the claims and the equivalents thereof.

### DETAILED DESCRIPTION

Embodiments of the present disclosure are directed to systems, methods, devices, and apparatuses for impeding and/or preventing vertebra displacement after a spinal fusion surgery. In various deployments described herein, the disclosure involves a spinal fusion instrumentation implanted into or onto the spine, and/or an apparatus attached to one or more vertebrae of the spine in order to impede and/or prevent vertebra displacement. The apparatus may be configured in terms one or more of materials, characteristics, size, and attachment features, such that lateral displacement of one or more vertebrae positioned vertically from the fused vertebrae is minimized, impeded, and/or prevented, thus facilitating successful and viable spinal correction surgery.

The details of some example embodiments of the systems, methods, apparatuses, and devices of the present disclosure are set forth in this description and in some cases, in other portions of the disclosure. Other features, objects, and advantages of the disclosure will be apparent to one of skill in the art upon examination of the present disclosure, description, figures, examples, and claims. It is intended that all such additional systems, methods, devices, apparatuses, features, and advantages be included within this description (whether explicitly or by reference), be within the scope of the present disclosure, and be protected by one or more of the accompanying claims.

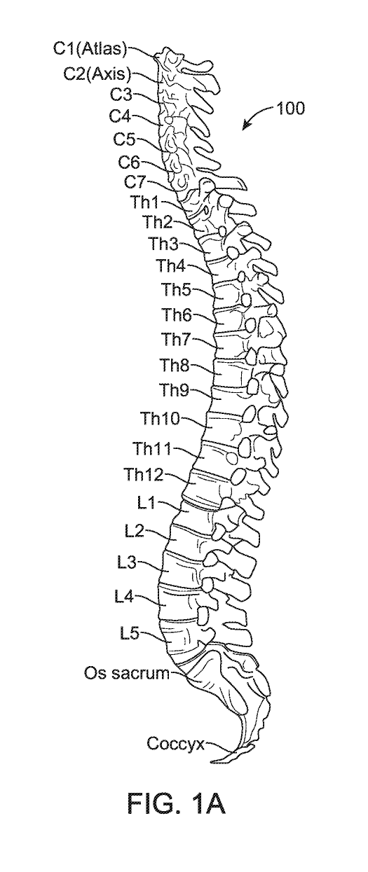

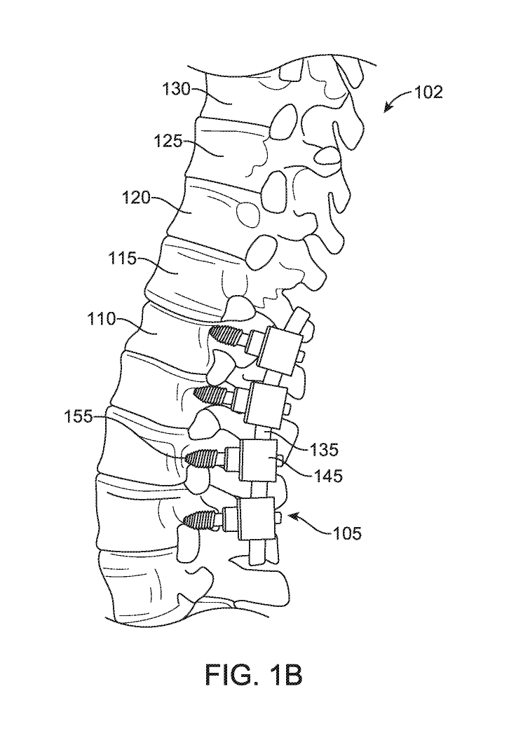

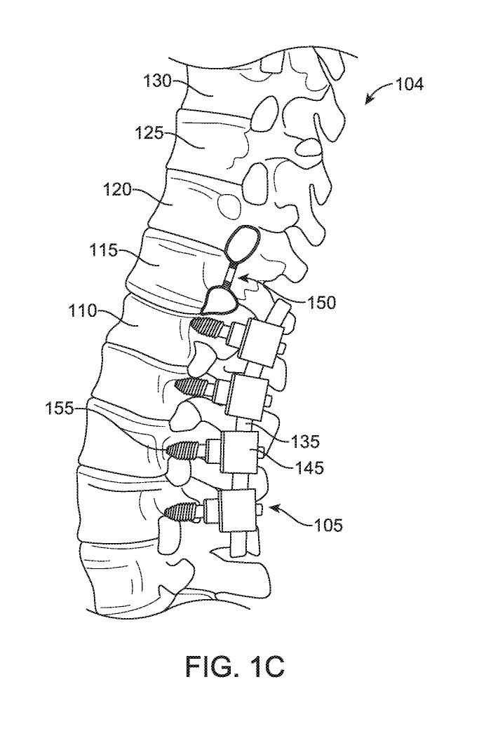

FIG. 1C is a diagram depicting a close-up side view of system 104 for impeding vertebra displacement. In FIG. 1C, the vertebral column, which includes vertebrae 110, 115, 120, 125, and 130, has undergone spinal fusion surgery. System 104 includes instrumentation 105 attached to a number of vertebrae, including vertebra 110, by pedicle screws 155, fixture 145, and support rod 135. As further illustrated in FIG. 1C, apparatus 150 is attached at one end to vertebra 110 and at the other end to vertebra 115. Positioned thusly, apparatus 150 minimizes, impedes, and/or prevents lateral displacement of one or more of vertebrae 115, 120, 125, and 130 following implantation of instrumentation 105. Namely, apparatus 150 provides lateral support for the vertebrae, and thus assists with resisting displacement forces. As will be described hereinbelow in further detail, and as will be appreciated by one of ordinary skill in the art upon studying the present disclosure, apparatus 150 may be assembled in various configurations, sizes, shapes, characteristics, and materials, and may be attached to the vertebral column in various manners, without departing from the spirit or scope of the present disclosure.

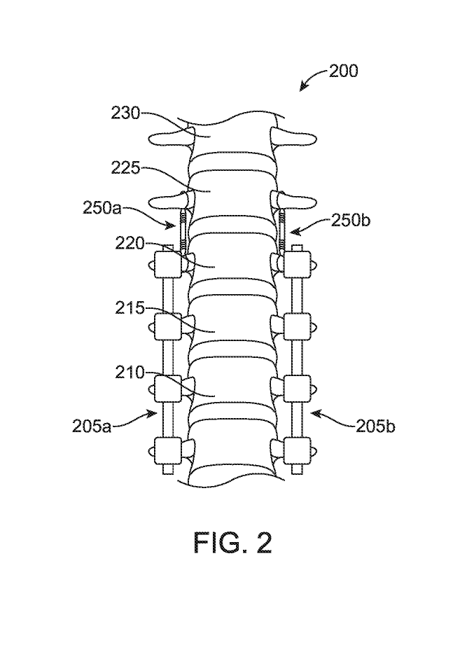

FIG. 2 is a diagram depicting a close-up posterior view of system 200 for impeding vertebra displacement. As shown, system 200 includes a spinal fusion taking the form of instrumentation 205a attached to one or more vertebrae, including vertebrae 210, 215, and 220, during spinal fusion surgery. The spinal fusion may be an implant inserted during spinal fusion surgery to promote a fusion of one or more of vertebrae 210, 215, and 220. Additionally, system 200 includes a spinal fusion taking the form of instrumentation 205b, which may be substantially similar to instrumentation 205a, and is affixed to the opposite side of the vertebral column. Instrumentation 205a, 205b may be substantially similar in nature to instrumentation 105.

System 200 also includes apparatus 250a, which is attached at one end to vertebra 220, and at the other end to vertebra 225. Similarly, apparatus 250b is attached to vertebrae 220 and 225 on the opposite side of the vertebral column. Apparatuses 250a and 250b are attached to vertebra 220 that is attached to instrumentation 205a and 205b, and are attached to vertebra 225 that is vertically adjacent to vertebrae 210, 215, and 220, to which instrumentation 205a and 205b are attached. Positioned thusly, apparatuses 250a and 250b minimize, impede, and/or prevent lateral displacement of vertebrae 220, 225, and/or 230 following implantation of instrumentation 205a and 205b. It will also be noted at this juncture, and described in further detail hereinbelow, that apparatuses 250a and 250b can be attached directly to instrumentation 205a or 205b, respectively.

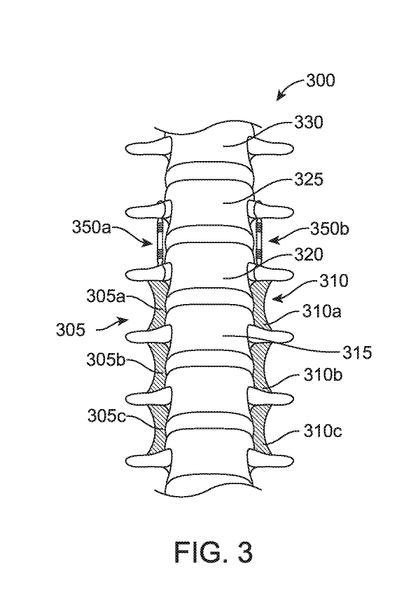

FIG. 3 is a diagram depicting a close-up posterior view of system 300 for impeding vertebra displacement. As shown, system 300 includes a spinal fusion taking the form of bone growths 305 and 310 that are attached to a number of vertebrae of the vertebral column, including vertebrae 315 and 320, following spinal fusion surgery. The spinal fusion may be present or may have grown due to an implant inserted during spinal fusion surgery. The spinal fusion may promote fusion of vertebrae 315 and 320. By way of example, bone growths may have been facilitated with a combination of one or more of bone grafts and instrumentation implanted during spinal fusion surgery. In some cases, once bone growths 305 and 310 have fused vertebrae 315 and 320, any implanted instrumentation (e.g., instrumentation 205a, 205b, with reference to FIG. 2) may be removed. As shown, bone growth 305 includes bone bridges 305a, 305b, and/or 305c that fuse vertically adjacent vertebrae (e.g., bone bridge 305a fuses vertebra 320 to vertebra 315). Bone growth 310 may likewise include bone bridges 310a, 310b, and/or 310c that fuse vertically adjacent vertebrae on the opposite side of the vertebral column.

System 300 also includes apparatus 350a, which is attached at one end to vertebra 325, and at the other end to vertebra 320. Similarly, apparatus 350b is attached to vertebra 320 and 325 on the opposite side of the vertebral column. Apparatuses 350a and 350b are attached to vertebra 320 that is attached to bone growths 305 and 310, and are attached to vertebra 325 that is vertically adjacent to vertebrae 315 and 320, to which bone growths 305 and 310 are attached. Positioned thusly, apparatuses 350a and 350b minimize, impede, and/or prevent lateral displacement of one or more of vertebrae 315, 320, 325, and/or 330 of the vertebral column, following the fusion of vertebra by way of bone growths 305 and 310. It will also be appreciated that apparatuses 350a and 350b can be attached directly to bone growths 305 and 310, respectively.

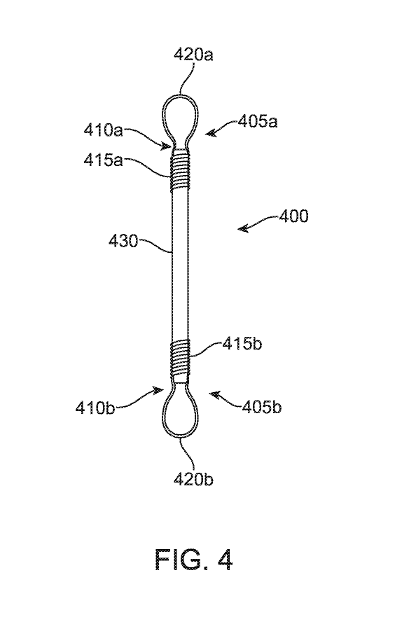

FIG. 4 is a diagram depicting example apparatus 400 for impeding vertebra displacement in accordance with various embodiments of the present disclosure. In some deployments, apparatus 400 is substantially similar to one or more of apparatuses 150, 250a, 250b, 350a, and 350b. As shown in FIG. 4, apparatus 400 includes tissue 430 with first end 410a and second end 410b.

Tissue 430 may be configured in various shapes, sizes, and types of tissue, including to have various characteristics and/or properties. For example, tissue 430 may be elongated in shape and may be a graft harvested from a living human (including, e.g., an autograft), may be cadaveric tissue harvested from a human cadaver (including, e.g., an allograft), or may be harvested from an animal. In alternative embodiments, tissue 430 includes, incorporates, or is formed from one or more non-tissue materials and/or synthetic materials (including, e.g., synthetic materials described in connection with first and second fasteners 405a and 405b below). As will be understood by one of skill in the art upon studying the present disclosure, tissue 430 may be selected so as to impede lateral displacement of vertically adjacent vertebrae after spinal fusion surgery. For example, tissue 430 may be selected in terms of material, size (length and thickness), source (e.g., body part, body type, physical or donor characteristics, etc.), and the like, so as to resist lateral force while allowing some level of flexibility in order to avoid shear or flexural damage to the tissues and/or damage to the vertebral column.

In embodiments, tissue 430 is replaced by an elongated member formed from non-tissue materials, such as the metal and/or synthetic materials described herein. In some cases, the elongated member may include a combination of tissue and non-tissue materials. The non-tissue materials may include, by way of example, one or more of any of the materials described herein with respect to fasteners etc. The elongated member may generally resemble the shape of tissue 430, but may be shaped and sized according to the patient's anatomy, the nature of the injury, and the like so as to resist lateral force while allowing some level of flexibility in order to avoid shear or flexural damage to the tissues and/or damage to the vertebral column. The elongated member may have a proximal end that, e.g., corresponds to first end 410a of tissue 430 and a distal end that, e.g., corresponds to second end 410b of tissue 430.

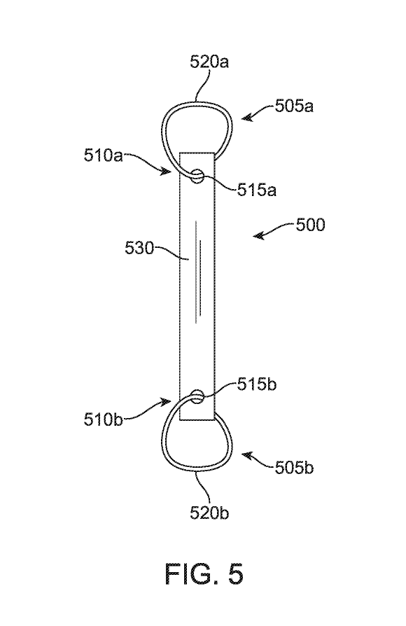

Apparatus 400 also includes first fastener 405a attached to first end 410a and configured to be coupled to a first vertebra (e.g., vertebra 225 shown in FIG. 2). First fastener 405a may include a first strand of material. Wrapping portion 415a of first fastener 405a may be wrapped around first end 410a. Alternatively or in addition, as will be discussed in detail with reference to FIG. 5, first fastener 405a may pass through first end 410a, e.g., via a hole or passage formed in first end 410a of tissue 430. Thusly, whether by wrapping portion 415a or by passing through first end 410a, or both in combination, first fastener 405a is secured to first end 410a. Adhesive may also be used to secure first fastener 405a to first end 410a. In some cases, first end 410a may include a bone chunk, and wrapping portion 415a may be wrapped around the bone chunk and/or first fastener 405a may pass through or be adhered to the bone chunk.

Apparatus 400 also includes second fastener 405b attached to second end 410b and configured to be coupled to a second vertebra (e.g., vertebra 220 shown in FIG. 2). Second fastener 405b may include a second strand of material, which may be substantially similar to the first strand of material described above. Wrapping portion 415b of second fastener 405b may be wrapped around second end 410b. Alternatively or in addition, as will be discussed in detail with reference to FIG. 5, second fastener 405b may pass through second end 410b, e.g., via a hole or passage formed in second end 410b of tissue 430. Thusly, whether by wrapping portion 415b or by passing through second end 410b, or both in combination, second fastener 405b is secured to second end 410b. Adhesive may also be used to secure first fastener 405a to first end 410a. In some cases, second end 410b may include a bone chunk, and wrapping portion 415b may be wrapped around the bone chunk and/or second fastener 405b may pass through or be adhered to the bone chunk.

In embodiments of apparatus 400, first and/or second fasteners 405a, 405b include first and/or second strands. First and second fasteners 405a and 405b, and/or more particularly the first and/or second strands, may be formed from various materials, such as synthetic or organic materials (e.g., tissue), and may be configured in various shapes and sizes. By way of example, first and second fasteners may include one or more of thread-like material, wire, flexible synthetic material, sutures, and organic material. Example materials include but are not limited to catgut suture, silk, nylon, polyester, PVDF, polypropylene, triclosan-coated sutures, polyglycolic acid, polylactic acid, polydioxanone, caprolactone, stainless steel or other metal (wires) with or without coatings, Polyetheretherketone (PEEK), titanium, carbon polymer, and cobalt-chrome. In addition, the first and/or second strands may be formed into various shapes (e.g., cross sectional profile being circular, oval, square, triangular, hexagonal, trapezoidal, etc.), sizes, and lengths, including based upon the anatomy or history of the patient or the nature of injury.

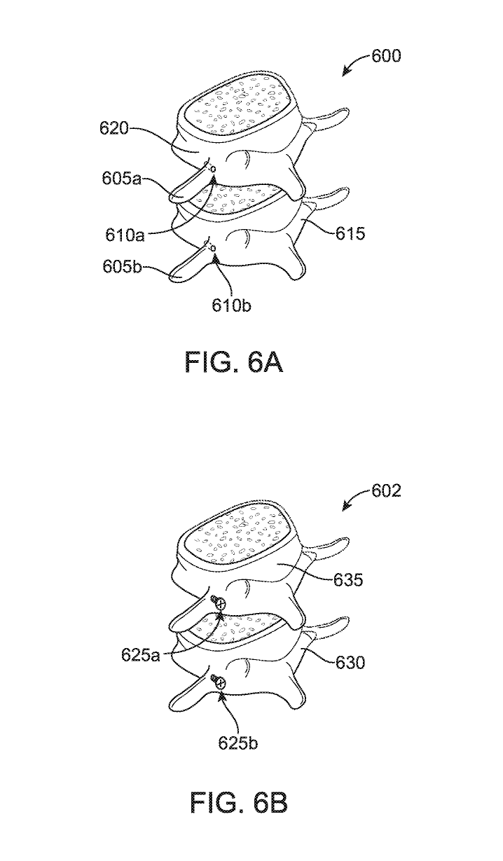

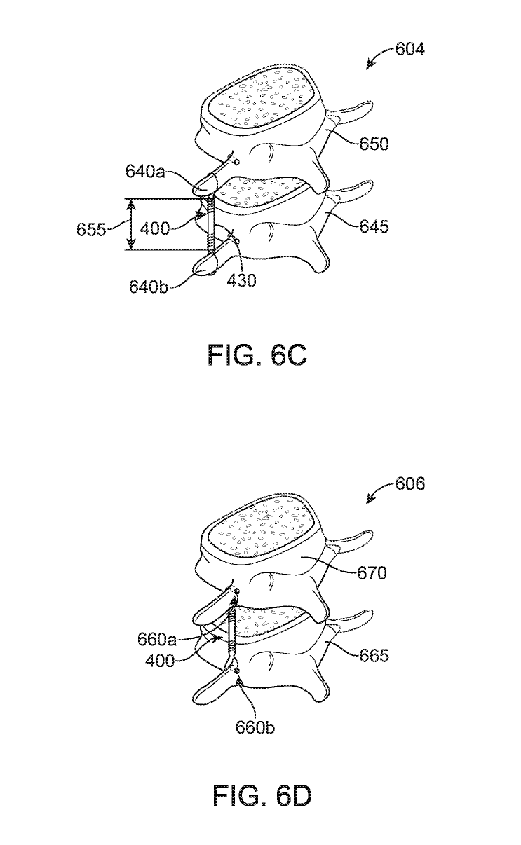

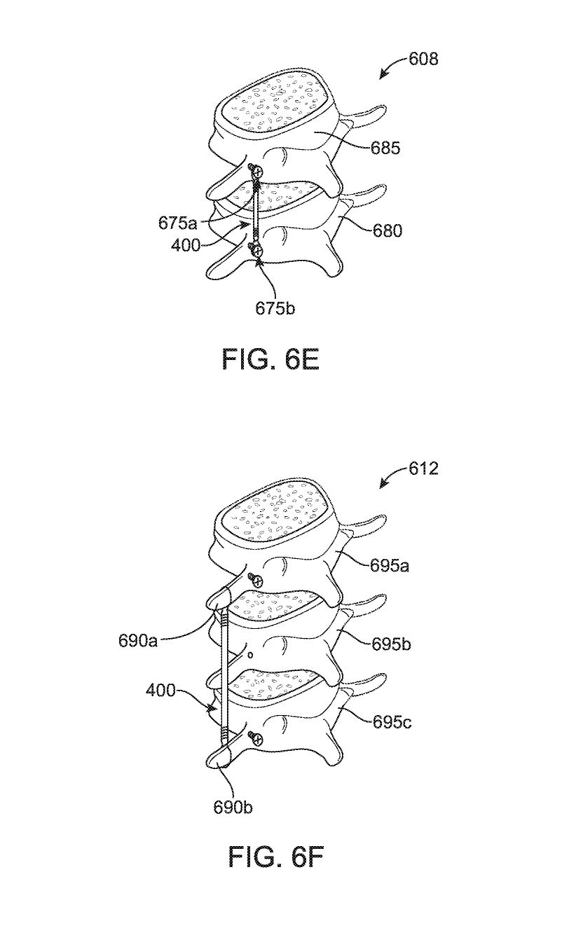

First and/or second fasteners 405a, 405b, in example deployments, include first and/or second loops 420a, 420b. In such deployments, first fastener 405a may include a first strand that may be formed into first loop 420a. Second fastener 405b may likewise include a second strand that may be formed into second loop 420b. First and second loops 420a and 420b are attachable to respective first and second vertebrae. By way of example, and as will be discussed in further detail in connection with FIGS. 6A-6F, a first anchor feature may be present in, on, or near a first vertebra, and a second anchor feature may be present in, on, or near a second vertebra. First fastener 405a may be configured to be coupled to the first vertebra via the first anchor feature, and second fastener 405b may be configured to be coupled to the second vertebra via the second anchor feature. Although not explicitly shown, first and second loops 420a and 420b may involve various looping, twisting, and/or knotting techniques with respect to first and second fasteners 405a and 405b. Such techniques may increase the security with which the first and second fasteners 405a and 405b are attached to tissue 430, and/or may facilitate coupling to the first/second anchor features.

Turning now to FIG. 5, apparatus 500 for impeding vertebra displacement after a spinal fusion surgery is shown, in accordance with embodiments of the present disclosure. As illustrated, apparatus 500 includes tissue 530 with first end 510a and second end 510b. Apparatus 500 also includes first fastener 505a and second fastener 505b. First fastener 505a includes first loop 520a, and second fastener 505b includes second loop 520b. First and second loops 520a and 520b may be formed in first and second strands included, respectively, in first and second fasteners 505a and 505b. As illustrated, in embodiments, first end 510a includes first passage 515a formed in first end 510a, and second end 510b includes second passage 515b formed in second end 510b. Further, in such embodiments, first fastener 505a may pass through first end 510a by way of first passage 515a, and second fastener 505b may pass through second end 510b by way of second passage 515b.

Generally speaking, aspects of apparatus 500 may be substantially similar to aspects of apparatus 400 unless otherwise noted. One difference alluded to above is that apparatus 500 includes first and second passages 515a and 515b. In this connection, it should be noted here the various ways that first and second loops 520a and 520b may be configured to interface with first and second passages 515a and 515b. In one example deployment, first fastener 505a may include a pre-formed loop of material. A first end of the pre-formed loop may be passed through first passage 515a and pulled out the other side thereof. Then, a second end of the pre-formed loop may be brought over first end 510a, such that the first end of the pre-formed loop may be pulled through the second end of the pre-formed loop and tightened around tissue 430 to form first loop 520a from the first end of th pre-formed loop. In another example deployment, where first fastener 505a includes a strand of material, a first end of the strand may passed through first passage 515a and attached to a second end of the strand (e.g., through knotting, adhesive, etc.), thus forming first loop 520a. Second loop 520b may be formed in similar fashions. In some cases, multiple loops may be formed in connection with each of first or second loops 520a or 520b, e.g., for redundancy purposes.

Further, as mentioned above in connection with FIG. 4, first and second passages 515a and 515b may be utilized in combination with wrapping first and/or second fasteners 505a, 505b around tissue 530, as well as with adhesive in some cases. In one example, a first end of first fastener 505a may be passed through first passage 515a and formed into a wrapping portion by being wrapped around first end 510a. First fastener 505a may then be formed into first loop 520a, and a second end of first fastener 505a may then be formed into another wrapping portion by being wrapped around first end 510a.

FIGS. 6A and 6B illustrate anchor features that may be used to connect an apparatus (e.g., apparatus 400 or 500) to a spine and impede vertebra displacement following spinal fusion surgery. FIGS. 6C, 6D, 6E, and 6F show the apparatus connected to the spine in various manners, including by the use of such anchor features. It will be appreciated upon studying these FIGS. that various anchor features may be mixed and matched as between the first and second vertebrae, and that combinations of anchor features may be used for a single vertebra. It will also be appreciated that different apparatuses can be mixed and matched along the length of the vertebral column, as well as with respect to the two sides of the vertebral column.

FIG. 6A illustrates vertebral column 600 that has been prepared for an apparatus for impeding vertebra displacement after a spinal fusion surgery to be attached thereto, in accordance with embodiments of the present disclosure. As shown, vertebral column 600 includes first vertebra 620 and second vertebra 615. First vertebra 620 includes first protrusion 605a, which in some instances is a transverse process of first vertebra 620. First protrusion 605a may in some cases be a fixation protrusion coupled to first vertebra 620 for an apparatus to be attached thereto. First vertebra 620 also includes first passage 610a formed in first vertebra 620. Generally speaking, first passage 610a may be formed by drilling or the like. In example implementations, first passage 610a is formed near the base of first protrusion 605a, but first passage 610a may be formed at any point along first protrusion 605a or may be formed nearer to the vertebral body of first vertebra 620. Likewise, second vertebra 615 includes second protrusion 605b, which in some instances is a transverse process of second vertebra 615. Second protrusion 605b may in some cases be a fixation protrusion coupled to second vertebra 615 for an apparatus to be attached thereto. Second vertebra 615 also includes second passage 610b formed in second vertebra 615. Second passage 610b may be formed at any point along second protrusion 605b or may be formed nearer to the vertebral body of second vertebra 615. As will be described in further detail herein, any one of first or second protrusion 605a or 605b, or first or second passages 610a or 610b, may be an example of an anchor feature.

Turning now to FIG. 6B, vertebral column 602 has been prepared for an apparatus for impeding vertebra displacement after a spinal fusion surgery to be attached thereto, in accordance with embodiments of the present disclosure. More specifically, vertebral column 602 includes first and second vertebra 635 and 630. First vertebra 635 includes first anchor feature 625a, and second vertebra 630 includes second anchor feature 625b. In the illustrated example embodiments, first and second anchor features 625a and 625b are formed from screws inserted into first and second vertebra 635 and 630. Other objects may be inserted into first and second vertebra 635 and 630 in order to form first and second anchor features 625a and 625b. Examples of such objects include nails, pins, and rods, whether formed synthetically or from metal material, as well as organic objects such as bone fragments or cartilage.

FIG. 6C illustrates vertebral column 604 with apparatus 400 attached between first and second vertebra 650 and 645, in accordance with embodiments of the present disclosure. As shown, apparatus 400 may be attached to first anchor feature 640a (e.g., by first fastener 405a or first loop 420a), which in this case is a first fixation protrusion (e.g., first protrusion 605a in FIG. 6A) coupled to first vertebra 650. Similarly, apparatus 400 may be attached to second anchor feature 640b (e.g., by second fastener 405b or second loop 420b), which in this case is a second fixation protrusion (e.g., second protrusion 605b in FIG. 6A) coupled to second vertebra 645. In embodiments, first loop 420a may be formed directly around first anchor feature 640a, in order to form a tight fit around first anchor feature 640a. Alternatively, during the formation of first loop 420a, first loop 420a may be attached to first anchor feature 640a after the first end of first fastener 405a is wrapped around first end 410a of tissue 430 but before the second end of first fastener 405a is wrapped around first end 410a. In this manner, first loop 420a may be tightened around the first anchor feature during the attachment of the first fastener to the first end of the tissue. Similar teachings may be applied with respect to second fastener 405b and the corresponding elements in connection with second anchor feature 640b.

Of course, apparatus 500 may be attached to vertebral column 604 by passing first and second loops 520a and 520b around first and second anchor features 640a and 640b, respectively. Additionally, apparatus 400 (or 500) may be attached to vertebral column 604 by looping a portion of first loop 420a (or 520a) around first anchor feature 640a and then passing apparatus 400 (or 500) through the first portion of first loop 420a (or 520a) and pulling first loop 420a (or 520a) tight around first anchor feature 640a. This teaching may be applied to second anchor feature 640b as well with respect to second fastener 405b (or 505b).

As is further illustrated in FIG. 6C, tissue 430 of apparatus 400 may be sized in terms of length to span distance 655 between first vertebra 650 and second vertebra 645 when apparatus 400 is attached to first vertebra 650 and second vertebra 645 (e.g., by first fastener 405a or first loop 420a, and by second fastener 405b or second loop 420b). By way of example, distance 655 may vary depending on the anatomy of the patient, the characteristics of the injury, the nature or characteristics of the first or second anchor feature 640a or 640b, and so on. By way of non-limiting example, where first and second vertebra 650 and 645 are vertically adjacent and successive vertebrae, distance 655 may range between about 10 mm or less and 150 mm or more for typical patients, but in some cases may be smaller or larger (e.g., depending on the patient's characteristics and the position of the vertebrae within the spinal column). This teaching may be applied to apparatus 500 as well. Of course, in cases where apparatus 400 or 500 is attached to non-successive vertebra (see, e.g., FIG. 6F), distance 655 may be greater.

FIG. 6D illustrates vertebral column 606 with apparatus 400 attached between first and second vertebra 670 and 665, in accordance with embodiments of the present disclosure. As shown, a first end of apparatus 400 may be attached to first anchor feature 660a (e.g., by first fastener 405a or first loop 420a), which in this case is a first passage (e.g., first passage 610a in FIG. 6A) formed in first vertebra 670. By way of specific example, first fastener 405a may pass through first anchor feature 660a. Similarly, a second end of apparatus 400 may be attached to second anchor feature 660b (e.g., by second fastener 405b or second loop 420b), which in this case is a second passage (e.g., second passage 610b in FIG. 6A) formed in second vertebra 665. Particularly, first fastener 405a may pass through first anchor feature 660a, and second fastener 405b may pass through second anchor feature 660b.

As described with respect to FIG. 6C, tissue 430 of apparatus 400 may be sized in terms of length to span a distance between first vertebra 670 and second vertebra 665. In embodiments, first loop 420a may be formed to pass through first anchor feature 660a, in order to attach apparatus 400 to first vertebra 670. For example, a first end of first fastener 405a may be wrapped around first end 410a. Then, during the formation of first loop 420a, the second end of first fastener 405a may be passed through first anchor feature 660a. Subsequently, the second end of first fastener 405a is wrapped around first end 410a. In this manner, first loop 420a may be tightened around first anchor feature 660a. Similar teachings may be applied with respect to second fastener 405b and the corresponding elements and second anchor feature 660b. Additionally, apparatus 400 (or 500) may be attached to vertebral column 606 by passing a portion of first loop 420a (or 520a) through first anchor feature 660a and then passing apparatus 400 (or 500) through the first portion of first loop 420a (or 520a) and pulling first loop 420a (or 520a) tight around first anchor feature 660a. This teaching may be applied to second anchor feature 660b as well with respect to second fastener 405b (or 505b).

FIG. 6E illustrates vertebral column 608 with apparatus 400 attached between first and second vertebra 685 and 680, in accordance with embodiments of the present disclosure. As shown, a first end of apparatus 400 is attached to first anchor feature 675a (e.g., by first fastener 405a or first loop 420a), which in this case is a first fixation protrusion (e.g., first anchor feature 625a in FIG. 6B), and more specifically is a screw in some embodiments, coupled to first vertebra 685. Similarly, a second end of apparatus 400 is attached to second anchor feature 675b (e.g., by second fastener 405b or second loop 420b), which in this case is a second fixation protrusion (e.g., second anchor feature 625b in FIG. 6B), and more specifically is a screw in some embodiments, coupled to second vertebra 680. By way of specific example, first and/or second fasteners 405a and/or 405b may loop around first and/or second anchor features 675a, 675b.

As described with respect to FIG. 6C, tissue 430 of apparatus 400 may be sized in terms of length to span a distance between first vertebra 685 and second vertebra 680. Of course, apparatus 500 may be attached to vertebral column 608 by passing first and second loops 520a and 520b around first and second anchor features 675a and 675b, respectively. Additionally, apparatus 400 (or 500) may be attached to vertebral column 608 by looping a portion of first loop 420a (or 520a) around first anchor feature 675a and then passing apparatus 400 (or 500) through the first portion of first loop 420a (or 520a) and pulling first loop 420a (or 520a) tight around first anchor feature 675a. This teaching may be applied to second anchor feature 675b as well with respect to second fastener 405b (or 505b).

FIG. 6F illustrates vertebral column 612 with apparatus 400 attached between first and second vertebra 695a and 695c, in accordance with embodiments of the present disclosure. As shown, a first end of apparatus 400 may be attached to first anchor feature 690a (e.g., by first fastener 405a or first loop 420a), which in this case is a first fixation protrusion (e.g., first protrusion 605a in FIG. 6A) coupled to first vertebra 695a. Similarly, a second end of apparatus 400 may be attached to second anchor feature 690b (e.g., by second fastener 405b or second loop 420b), which in this case is a second fixation protrusion (e.g., second protrusion 605b in FIG. 6A) coupled to second vertebra 695c. In FIG. 6F, apparatus 400 is connected at opposite ends to first and second vertebra 695a and 695c, and vertebral column 612 includes one or more interposing vertebrae 695b between first and second vertebra 695a and 695c. As described with respect to FIG. 6C, tissue 430 of apparatus 400 may be sized in terms of length to span a distance between first vertebra 695a and second vertebra 695c, including when vertebra column 612 includes one or more interposing vertebrae 695b. Of course, apparatus 500 may be attached to vertebral column 612 in the manner described above in connection with FIGS. 6C-6E, and different anchor features may be employed in connection with FIG. 6F.

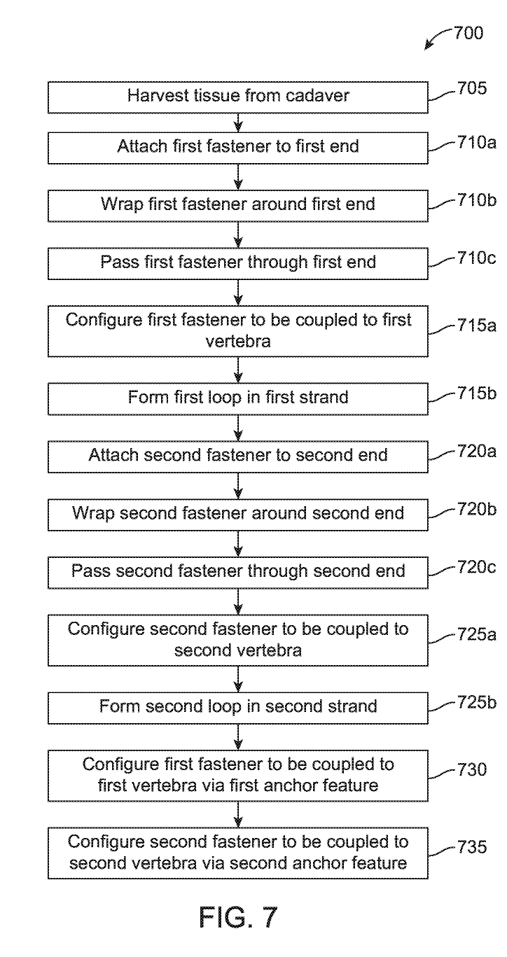

FIGS. 7-10 illustrate operational flow diagrams depicting various operations of methods 700, 800, 900, and 1000, respectively, and accompanying embodiments related to impeding vertebra displacement after spinal fusion surgery. Generally, methods 700, 800, 900, and 1000 relate to coupling a tissue or other object between vertebrae of a vertebral column, where nearby vertebrae have been fused via spinal fusion surgery. The operations of the various methods described herein are not necessarily limited to the order described or shown in the figures, and one of skill in the art will appreciate, upon studying the present disclosure, variations of the order of the operations described herein that are within the spirit and scope of the disclosure.

The operations and sub-operations of methods 700, 800, 900, and 1000 may be carried out, in some cases, by one or more of the components, elements, devices, features, and/or apparatuses described and/or referenced in FIGS. 1C, 2, 3, 4, 5, and 6A-F. In such instances, the description of methods 700, 800, 900, and 1000 may refer to a corresponding component, element, etc., but regardless of whether an explicit reference is made, one of skill in the art will recognize, upon studying the present disclosure, when the corresponding component, element, etc. may be used to carry out one or more aspects of the method. Further, it will be appreciated that such references do not necessarily limit the described methods to the particular component, element, etc. referred to. Thus, it will be appreciated by one of skill in the art upon studying the present disclosure that aspects and features described above in connection with (sub-) components, elements, devices, and features, etc., including variations thereof, may be applied to the various operations described in connection with methods 700, 800, 900, and/or 1000 without departing from the scope of the present disclosure.

Turning now to FIG. 7, embodiments of method 700 for impeding vertebra displacement after a spinal fusion surgery are illustrated, in accordance with the present disclosure. More specifically, embodiments of method 700 involve attaching fasteners to an elongated tissue, and configuring the fasteners to be coupled to vertebrae, one or more of which has been or is to be coupled to one or more other vertebrae via spinal fusion instrumentation. In this manner, the operations of method 700 increase the effectiveness of spinal fusion surgery.

At operation 705, method 700 optionally includes harvesting an elongated tissue from a cadaver. The elongated tissue, in example deployments, may be substantially similar to tissue 430 and/or 530, described hereinabove. Many variations are possible in relation to operation 705. By way of illustration, and as alluded to above, the elongated tissue in some cases may be substituted and/or augmented by tissue of different shapes, including shapes not elongated in nature—e.g., unelongated, shortened, or various other shapes. In other examples, portions of the tissue are elongated while other portions may be characterized as unelongated or shortened. Furthermore, regardless of the nature of the tissue in terms of shape/size, the elongated tissue may be harvested from a living person, who could be the person receiving the implant or another living person, or could be harvested from a human cadaver (e.g., autograft, isograft, or allograft could be used). In example implementations of the present disclosure, portions of the tissue are harvested from a living human and combined with portions harvested from a human cadaver. Additionally, such portions may be combined with tissue harvested from living or cadaveric animals (e.g., pig, cow, or other suitable animal), or animal tissue alone may be used (xenograft).

Once the tissue or elongated tissue is harvested, the tissue may be configured for implantation. In this connection, at operation 710a, method 700 includes attaching a first fastener to a first end of the tissue. With reference to FIGS. 4 and 5, examples of operation 710a may involve attaching first fastener 405a (or 505a) to first end 410a (or 510a) of tissue 430 (or 530). Embodiments of method 700 further include operation 710b, which entails wrapping the first fastener around the first end of the tissue. By way of example and with reference to FIG. 4, operation 710b may include wrapping a portion of first fastener 405a around first end 410a, e.g., to form wrapping portion 415a. Alternatively or additionally to operation 710b, method 700 may include, at operation 710c, passing the first fastener through the first end of the tissue. With reference to FIG. 5, examples of operation 710c may involve passing first fastener 505a through first end 510a of tissue 530 via first passage 515a. The first fastener may include a first strand that may be wrapped around and/or passed through the first end of the tissue (e.g., in accordance with operations 710b, 710c). The first strand may be formed from various materials, as described above, e.g., with reference to FIG. 4. In example embodiments of method 700, the first strand includes one or more of thread-like material, wire, and flexible synthetic material.

With further reference to FIG. 7, at operation 715a, method 700 includes configuring the first fastener to be coupled to a first vertebra. By way of example, as shown in FIG. 2, the first end of apparatus 250a is configured to be attached (and is attached in FIG. 2) to first vertebra 225. In embodiments of method 700, configuring the first fastener to be coupled to a first vertebra includes, at operation 715b, forming a first loop from the first strand of the first fastener. Thusly, the first strand may include a first loop attachable to the first vertebra. With reference to FIGS. 4, 5, and 6C, first fastener 405a (or 505a) of apparatus 400 (or 500) may be configured to be coupled to first vertebra 650 by first loop 420a (or 520a) formed in first fastener 405a (or 505a), and more specifically the first strand thereof. As described above with reference to FIGS. 6A-6F, the first fastener may be attachable and/or attached to the first vertebra in a number of ways and using a number of mechanisms.

At operation 720a, method 700 involves attaching a second fastener to a second end of the tissue. In many respects, operation 720a may be substantially similar to operation 710a. With reference to FIGS. 4 and 5, examples of operation 720a may involve attaching second fastener 405b (or 505b) to second end 410b (or 510b) of tissue 430 (or 530). Embodiments of method 700 further include operation 720b, which entails wrapping the second fastener around the second end of the tissue. By way of example and with reference to FIG. 4, operation 720b may include wrapping a portion of second fastener 405b around second end 410b, e.g., to form wrapping portion 415b. Alternatively or additionally to operation 720b, method 700 may include, at operation 720c, passing the second fastener through the second end of the tissue. With reference to FIG. 5, examples of operation 720c may involve passing second fastener 505b through second end 510b of tissue 530 via second passage 515b. The second fastener may include a second strand that may be wrapped around and/or passed through the second end (e.g., in accordance with operations 720b, 720c). The second strand may be formed from various materials, e.g., as described above with reference to FIG. 4 and/or in connection with the first strand.

With further reference to FIG. 7, at operation 725a, method 700 includes configuring the second fastener to be coupled to a second vertebra. The second vertebra is coupled to a third vertebra via a spinal fusion. For example, as shown in FIG. 2, the first end of apparatus 250a is configured to be attached (and is attached in FIG. 2) to first vertebra 225. The second end of apparatus 250a is configured to be attached (and is attached in FIG. 2) to second vertebra 220. Second vertebra 220 is attached to third vertebra 215 (and in this case to vertebra 210 as well) via instrumentation 205a and 205b. In embodiments, the second fastener may be configured to be coupled to the second vertebra by attaching to directly the spinal fusion rather than to the second vertebra.

In instances of method 700, configuring the second fastener to be coupled to the first vertebra includes, at operation 725b, forming a second loop from the second strand of the second fastener. Thusly, the second strand may include a second loop attachable to the second vertebra. With reference to FIGS. 4, 5, and 6C, second fastener 405b (or 405b) of apparatus 400 (or 500) may be configured to be coupled to second vertebra 645 by second loop 420b (or 520b) formed in second fastener 405b (or 505b), and more specifically the second strand thereof. As described above with reference to FIGS. 6A-6F, the second fastener may be attachable and/or attached to the second vertebra in a number of ways and using a number of mechanisms. In embodiments in which the second fastener is configured to be coupled to the second vertebra by attaching to directly the spinal fusion rather than to the second vertebra, the second loop may be formed or manipulated so as to surround a portion of the spinal fusion, including in some example an anchor feature of the spinal fusion.

Method 700 may also include, at operation 730, configuring the first fastener to be coupled to the first vertebra via a first anchor feature. Examples of anchor features are described above, e.g., in connection with FIGS. 6A and 6B. Accordingly, operation 730 typically involves configuring the first fastener to be coupled to a first fixation protrusion, screw, and/or passage in the first vertebra, or other such anchor feature or combination of features. By way of illustration, and with reference to FIGS. 4, 5, and 6C-6E, operation 730 may entail configuring first fastener 405a (or 505a) of apparatus 400 (or 500) to be coupled to first vertebra 650 (or 670 or 685) via first anchor feature 640a (or 660a or 675a). In some cases, it will be appreciated that, e.g., where the first anchor feature is a first passage formed in the first vertebra, the first loop will not be formed until after the first strand is passed through the first passage. Similarly, where, e.g., the first anchor feature is a first fixation protrusion (e.g., bone or screw), the first loop may be formed before the first fastener is coupled to the first anchor feature. Whether the first anchor includes a first passage or a first fixation protrusion, a portion of the first loop may be passed through the passage or looped around the fixation protrusion, and the apparatus (400 or 500) may be passed through the portion of the first loop such that the first loop may be tightened.

With continued reference to FIG. 7, method 700 may also include, at operation 735, configuring the second fastener to be coupled to the second vertebra via a second anchor feature. Operation 735 may be substantially similar to operation 730 in many cases. Nevertheless, it should be noted at this juncture that the first and second anchor features need not be the same, and hence, operations 730 and 735 may be different in some ways. By way of illustration, and with reference to FIGS. 4, 5, and 6C-6E, operation 730 may entail configuring second fastener 405b (or 505b) of apparatus 400 (or 500) to be coupled to second vertebra 645 (or 660 or 680) via second anchor feature 640b (or 660b or 675b). As mentioned, the first and second anchor features may be different—e.g., the first anchor feature may include a passage formed in the first vertebra, whereas the second anchor feature may include a screw inserted in the second vertebra. And operation 735 in such an instance may differ from operation 730. Additionally, as mentioned above, the second fastener may be configured to be coupled to the second vertebra by attaching to directly the spinal fusion rather than to the second vertebra. In such example implementations, the second loop may be formed before the attachment is made or during the attachment, e.g., depending on the anchor feature of the spinal fusion. The anchor feature of the spinal fusion may include a rod thereof, a bone bridge, a screw, a latch, a hook, a passage therein, or any type of fixation protrusion or mechanism.

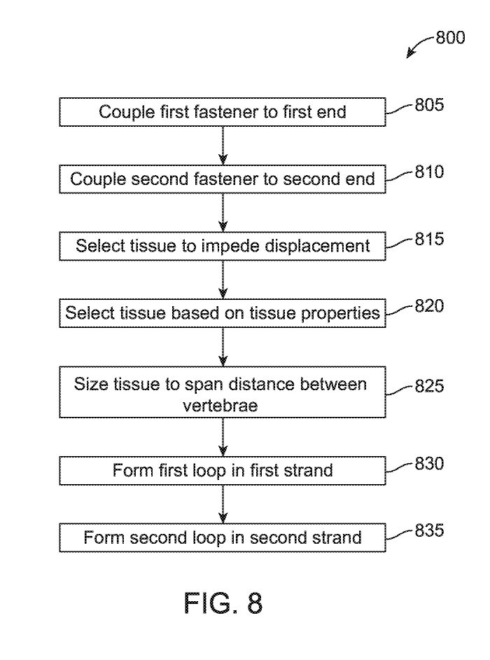

Turning now to FIG. 8, the operations of method 800 may be used in connection with spinal fusion surgery in order to reduce, impede, or prevent lateral displacement of vertically adjacent vertebrae. More specifically, in embodiments of method 800, fasteners are coupled to a tissue, where the fasteners are attachable to vertically adjacent vertebrae. With the tissue attached to the vertically adjacent vertebrae using the fasteners, the tissue provides lateral support and some resistivity to impede relative displacement of the vertically adjacent vertebrae when at least one of the vertically adjacent vertebrae has been or will be coupled to other vertebrae by way of a spinal fusion. In this manner, the operations of method 800 increase the effectiveness of spinal fusion surgery.

At operation 805, method 800 includes coupling a first fastener to a first end of a tissue. To illustrate, with reference to FIGS. 4 and 5, first fastener 405a (or 505a) may be coupled to first end 410a (or 510a) of tissue 430 (or 530). There are a number of ways in which operation 805 may be carried out. One example involves wrapping a portion of the first fastener around the first end of the tissue. As shown in FIG. 4, wrapping portion 415a may be used to couple first fastener 405a to first end 410a of tissue 430. Another example involves passing the first fastener through the tissue. FIG. 5 illustrates that this may be done by passing first fastener 505a through first end 510a of tissue 530 via passage first 515a. Additional examples include the first fastener being adhered and/or fused to the tissue. These coupling techniques need not be used in isolation, but may be combined in various ways (e.g., wrapping, passing through, and/or adhesion, etc.).

At operation 810, method 800 includes coupling a second fastener to a second end of the tissue. By way of example and with reference to FIGS. 4 and 5, second fastener 405b (or 505b) may be coupled to second end 410b (or 510b) of tissue 430 (or 530). This may be done in substantially similar fashions as described above in connection with operation 805, including wrapping around and/or passing through the tissue, as well as other means described above. Nevertheless, it will be appreciated that the first and second fasteners need not be coupled to the tissue in the same manner, and that multiple coupling techniques described herein may be mixed and matched as between the first and second fasteners.

With respect to method 800, the first and second fasteners are attachable to vertically adjacent vertebrae in a vertebral column. At least one of the vertically adjacent vertebrae is coupled to an other vertebra of the vertebral column by way of a spinal fusion. As described above, this may involve one or more of a spinal fusion instrumentation, a bone growth, and/or bone grafts. In this manner, the tissue may be coupled two vertically adjacent vertebrae and may be used in connection with spinal fusion surgery to improve the effectiveness thereof. The tissue is selected so as to impede a lateral displacement of, e.g., the first of the vertically adjacent vertebrae relative to the second of the vertically adjacent vertebrae, when the second vertebra has been coupled to one or more of the other vertebrae by way of a spinal fusion. The selection of the tissue may thus involve a selection of one or more of the tissue's size, shape, properties, nature, source, and materials.

In example implementations, method 800 includes, at operation 815, selecting the tissue so as to impede a lateral displacement of the first of the vertically adjacent vertebrae relative to the second of the vertically adjacent vertebrae. Operation 815 may involve selecting the tissue based on one or more of the tissue's size, shape, source, properties, nature, and materials, and/or one or more of the nature of the injury, and the patient's size, dimensions, weight, age, lifestyle, past history, characteristics, and the like. By way of example, for a heavier or older patient, a stronger or more rigid tissue may be selected for impeding lateral displacement. For a younger or more athletic patient, a more flexible piece of tissue may be selected. If the patient has previously experienced recovery problems following a spinal fusion surgery, a stronger, more rigid piece of tissue may be selected. In embodiments, multiple tissues may be attached to vertically adjacent vertebrae, such that the resistance profile of the transition between fused and non-fused vertebrae may be lengthened out and tapered or otherwise varied across several vertebrae. In such embodiments, each of the tissues may be selected per operation 815 in order to create a desired resistance profile.

With further reference to FIG. 8, method 800 may include, at operation 820, selecting the tissue based on properties of the tissue that facilitate impedance of the lateral displacement. Operation 820 can in some ways be considered a subset of operation 815, in which the selection is based on the tissue properties, apart from properties of the patient or the injury. With this note, aspects of the above description of operation 815 can be applied to operation 820. To further expound, the tissue may be harvested from different sources depending on the desired characteristics. In some cases, the tissue harvested may be an Achilles tendon, an anterior cruciate ligament, an anterolateral ligament, a medial collateral ligament, a posterior cruciate ligament, a lateral collateral ligament, or any other ligament, tendon, and/or combination of the same with bone and/or cartilage. Moreover, the tissue may be selected based on the characteristics of the tissue as determined by characteristics of the tissue donor—e.g., one or more of the donor's age, gender, lifestyle, past history, etc. The above characteristics of the tissue may all relate to facilitation of impedance of the lateral displacement of vertically adjacent vertebrae, following a spinal fusion surgery as described herein.

At operation 825, method 800 may include sizing a length of the tissue to span a distance between the vertically adjacent vertebrae when the first fastener is attached to the first of the vertically adjacent vertebrae and the second fastener is attached to the second of the vertically adjacent vertebrae. By way of illustration and with reference to FIGS. 4 and 6C, the length of tissue 430 (of apparatus 400) may be sized so as to span distance 655 between first vertebra 650 and second vertebra 645 when first fastener 405a is attached to first anchor feature 640a and second fastener 405b is attached to second anchor feature 640b. Of course, the sizing of the length may be varied to accommodate various scenarios, such as variations in the distance between the vertically adjacent vertebrae, one or more interposing vertebrae (e.g., as shown in FIG. 6F), variations in the anchor features used for attaching the tissue (e.g., if one end of the tissue is attachable and/or attached to the spinal fusion), etc. It will also be appreciated that this teaching can be applied in connection with apparatus 500, and/or in connection with the various types of anchor features described herein.

Operation 825 may be carried out at many different points in time with respect to the spinal fusion surgery. The sizing may occur, for example, during the harvesting of the tissue (e.g., by the incisions or other mechanism for removing the tissue from the source), before the fasteners are coupled to the tissue, or iteratively after the fasteners are coupled to the tissue. In some cases, the sizing occurs during the surgery in which the tissue is to be attached to the vertebral column. This surgery may be part of the spinal fusion surgery, or may be part of a separate procedure carried out thereafter or beforehand. One example of the iterative approach is to attach one or both of the first or second fasteners to the tissue, and then determine the length of the tissue to enable the tissue to span the distance between the vertebrae. If necessary, one or both of the first or second fasteners may be removed from the tissue such that the tissue can be resized, and then the fasteners can be re-coupled to the tissue. Typically, the tissue is shortened, but the fasteners may be re-positioned so as to increase the distance between the attachment points of the tissue to the vertebrae (effectively lengthening the portion of the tissue spanning between the vertebrae).

In example deployments of the present disclosure, method 800 includes, at operation 830, forming a first loop in a first strand of the first fastener. In such deployments, the first fastener includes a first strand with a first loop attachable to a first protrusion that protrudes from the first of the vertically adjacent vertebrae. For example, as shown in FIGS. 4, 5, 6C, 6E, and 6F, first fastener 405a (or 505a) includes first loop 420a (or 520a) that may be formed from the first strand. First loop 420a (or 520a) is attachable to first anchor feature 640a (or 675a or 690a). For example, first loop 420a (or 520a) may be attached around first protrusion 640a (or 675a or 690a). With reference to FIG. 6D, a portion of first loop 420a (or 520a) may pass through first anchor feature 660a, and then apparatus 400 (or 500) may be passed through the portion such that first loop 420a (or 520a) may be tightened and attached to first vertebra 670. This technique may also be applied to FIGS. 6C, 6E, and/or 6F as well.

Additional example deployments include, at operation 835, forming a second loop in a second strand of the second fastener. The second fastener in such deployments includes a second strand with a second loop attachable to a second protrusion that protrudes from the second of the vertically adjacent vertebrae. For example, as shown in FIGS. 4, 5, 6C, 6E, and 6F, second fastener 405b (or 505b) includes second loop 420b (or 520b) that may be formed from the second strand. Second loop 420b (or 520b) is attachable to second anchor feature 640b (or 675b in FIG. 6E, or 690b in FIG. 6F), including using the techniques described above in connection with operation 830.

In embodiments of method 800, the spinal fusion includes one or more of a bone graft, a spinal fusion instrumentation, and a bone growth. In some cases, one or more of the first and second fasteners is attachable to the bone graft, the spinal fusion instrumentation, and/or the bone growth. Specifically, if the instrumentation includes rods and/or pedicle screws, the first and/or second fasteners may be configured to be attached thereto. If the instrumentation includes bone growths, such as, e.g., bone growths 305 and 310 shown in FIG. 3, the first and/or second fasteners may be configured to be attached thereto (e.g., to be looped around one of the bone bridges thereof).

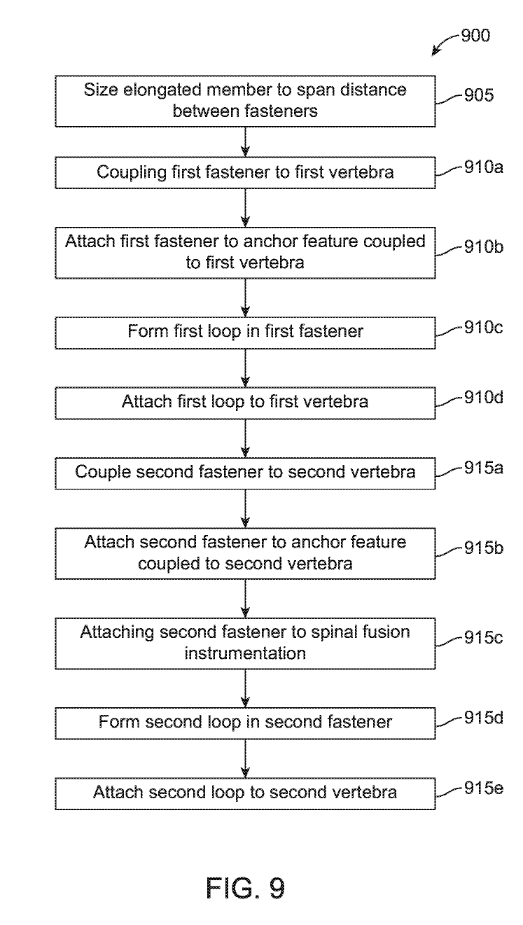

Turning now to FIG. 9, method 900 for impeding vertebra displacement using an elongated member is provided. More specifically, in embodiments of method 900, fasteners are coupled to first and second vertebrae. In this connection, a first fastener is attached to a proximal end of the elongated member, and a second fastener is attached to a distal end of the elongated member. In some examples, the elongated member is substantially similar to tissue 430 or tissue 530. In some cases, the elongated member may be formed at least partially from non-tissue material, such as synthetic or other materials, including various materials described herein, e.g., in connection with fasteners etc. An implant is coupled between the second vertebra and a third vertebra and configured to promote spinal fusion. For example, the implant may include a spinal instrumentation, a bone graft, and/or a bone growth. By providing additional lateral support to the vertebral column near or around the implant, the operations of method 900 increase the effectiveness of spinal fusion surgery.

As shown in FIG. 9, method 900 optionally includes, at operation 905, sizing the elongated member to span a distance between the first and second fasteners when the first fastener is coupled to the first vertebra and the second fastener is coupled to the second vertebra. As with operation 825, operation 905 may occur at different points in time and in different sequential orders with respect to the other operations of method 900. In example implementations of method 900, the first and second vertebrae are vertically adjacent and/or the second and third vertebrae are vertically adjacent. In additional implementations, one or more vertebrae interpose the first and second vertebrae and/or the second and third vertebrae. To illustrate one example of how operation 905 may be used in connection with method 900, FIG. 6C shows distance 655 between first and second fasteners 405a and 405b when first fastener 405a is coupled to first vertebra 650 and second fastener 405b is coupled to second vertebra 645 (thus attaching apparatus 400 therebetween). Of course, this teaching may be applied to apparatus 500 as well as to the arrangements shown FIGS. 6D-6F.

At operation 910a, method 900 includes coupling the first fastener to a first vertebra. The first fastener is attached to the proximal end of the elongated member. By way of example, with reference to FIGS. 4, 5, 6C-6F, operation 910a may involve coupling first fastener 405a (or 505a) to first vertebra 650 (or 670, 685, or 695a), where first fastener 405a (or 505a) is attached to first end 410a (or 510a) of tissue 430 (or 530). This coupling and attachment may be effected in a fashion substantially similar to that described above in connection with methods 700 and/or 800.

In example implementations of method 900, coupling the first fastener to the first vertebra includes, at operation 910b, attaching the first fastener to an anchor feature coupled to the first vertebra. By way of illustration and with reference to FIGS. 4, 5, and 6C-6F, first fastener 405a (or 505a) of apparatus 400 (or 500) may be attached to first anchor feature 640a (or 660a, 675a, or 690a). In embodiments of method 900, the first fastener includes a first loop. The first loop may be formed at operation 910c (e.g., in a fashion substantially similar to operations 715b and/or 830). In some such embodiments, coupling the first fastener to the first vertebra includes, at operation 910d, attaching the first loop to the first vertebra. For example, the first loop may be attached to a first fixation protrusion that protrudes from the first of the vertically adjacent vertebrae, or to a passage or other anchor feature in the first of the vertically adjacent vertebrae. One way this may be done, as shown in FIGS. 4, 5, and 6C-6F, is that first loop 420a (or 520a) may be attached to first protrusion 640a (or 675a or 690a). Alternatively or in addition, first loop 420a (or 520a) may be passed through first anchor feature 660a.

With further reference to FIG. 9, at operation 915a, method 900 includes coupling a second fastener to a second vertebra. The second fastener is attached to the distal end of the elongated member. By way of example, with reference to FIGS. 4, 5, 6C-6F, operation 915a may involve coupling second fastener 405b (or 505b) to second vertebra 645 (or 665, 680, or 695c), where second fastener 405b (or 505b) is attached to second end 410b (or 510b) of tissue 430 (or 530). This coupling and attachment may be effected in a fashion substantially similar to that described above in connection with methods 700 and/or 800.

At operation 915b, method 900 includes attaching the second fastener to a second anchor feature coupled to the second vertebra. In some embodiments of operation 915b, the second anchor feature is not part of the spinal fusion instrumentation, but is coupled to the second vertebra by other means, as is described above with reference to FIGS. 4, 5, 6A-6F, 7 and/or 8, hereinabove. By way of illustration and with reference to FIGS. 4, 5, and 6C-6F, second fastener 405b (or 505b) of apparatus 400 (or 500) may be attached to second anchor feature 640b (or 660b, 675b, or 690b). In example implementations of operation 915b, the implant is a spinal fusion instrumentation, and, at operation 915c, method 900 includes attaching the second fastener to the spinal fusion instrumentation. In such cases, the second anchor feature may be part of the spinal fusion instrumentation, which is in turn coupled to the second vertebra.

In embodiments of method 900, the second fastener includes a second loop. The second loop may be formed at operation 915d (e.g., in a fashion substantially similar to operations 715b and/or 830). In some such embodiments, coupling the second fastener to the second vertebra includes, at operation 915e, attaching the second loop to the second vertebra. For example, the second loop may be attached to a second fixation protrusion that protrudes from the first of the vertically adjacent vertebrae, or to a passage or other anchor feature in the second of the vertically adjacent vertebrae. One way this may be done, as shown in FIGS. 4, 5, and 6C-6F, is that second loop 420b (or 520b) may be attached to second anchor feature 640b (or 675b or 690b). Alternatively or in addition, second loop 420b (or 520b) may be passed through second anchor feature 660b.

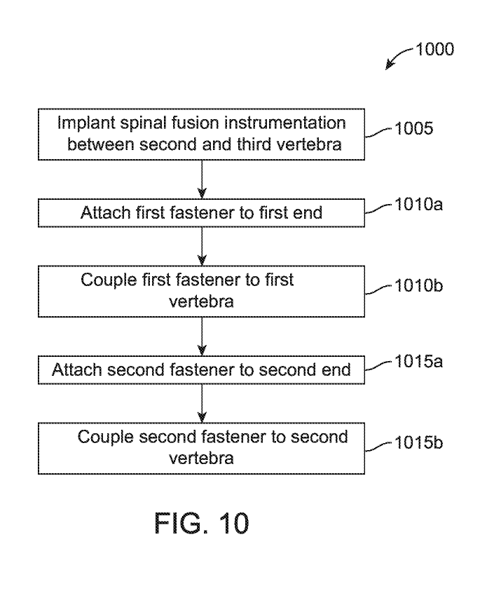

Turning now to FIG. 10, method 1000 for performing a spinal fusion surgery is provided, in accordance with embodiments of the present disclosure. Generally, embodiments of method 1000 involve implanting a spinal fusion instrumentation between vertebrae and coupling tissue between vertebrae adjacent the spinal fusion instrumentation, such that the tissue assists in impeding lateral displacement of the vertebrae after the spinal fusion surgery.

More specifically, at operation 1005, method 1000 includes implanting a spinal fusion instrumentation between a second vertebra and a third vertebra. The spinal fusion instrumentation is implanted in this fashion so as to promote a fusion between the second and third vertebrae. By way of example and with reference to FIG. 2, instrumentation 205a and 205b may be implanted between second vertebra 220 and third vertebra 215 (as well as vertebra 210 in some cases, and potentially between other vertebrae as well). FIG. 1C may also provide illustrative in this regard. With reference to FIG. 3, in embodiments, bone grafts may be implanted between the second and third vertebrae, as well as other vertebrae of the depicted vertebral column. This may be done in lieu of or in addition to the implantation of spinal fusion instrumentation.

Referring back to FIG. 10, method 1000 optionally includes, at operation 1010a, attaching a first fastener to a first end of an elongated tissue. To illustrate with reference to FIGS. 4 and 5, first fastener 405a (or 505a) may be attached to first end 410a (or 510a) of tissue 430 (or 530). In embodiments, operation 1010a is substantially similar to or involves aspects of operations 710a, 710b, 710c, and/or 805. Method 1000 includes, at operation 1010b, coupling the first fastener to a first vertebra that is not coupled to the second vertebra or the third vertebra via the spinal fusion. For example, with reference to FIGS. 4, 5, and 6C-6F, operation 1010b may involve coupling first fastener 405a (or 505a) to first anchor feature 640a (or 660a, 675a, or 690a) of first vertebra 650 (or 670, 685, or 695a).

At operation 1015a, method 1000 optionally includes attaching a second fastener to a second end of the elongated tissue. To illustrate with reference to FIGS. 4 and 5, second fastener 405b (or 505b) may be attached to second end 410b (or 510b) of tissue 430 (or 530). In embodiments, operation 1015a is substantially similar to or involves aspects of operations 720a, 720b, 720c, and/or 810. Method 1000 includes, at operation 1015b, coupling the second fastener to the second vertebra. For example, with reference to FIGS. 4, 5, and 6C-6F, operation 1015b may involve coupling second fastener 405b (or 505b) to second anchor feature 640b (or 660b, 675b, or 690b) of second vertebra 645 (or 665, 680, or 695c). Coupled to the first and second vertebrae, the elongated tissue impedes a lateral displacement of the first vertebra after spinal fusion surgery. In connection with method 1000, the elongated tissue may be selected according to the above-described criteria so as to more effectively impede the lateral displacement of vertebrae.

Various embodiments have been described with reference to specific example features thereof. It will, however, be evident to one of ordinary skill in the art upon studying the present disclosure that various modifications and changes may be made to such embodiments without departing from the broader spirit and scope of the various embodiments as set forth in the appended claims. The specification and figures are, accordingly, to be regarded in an illustrative rather than a restrictive sense.

Moreover, although described above in terms of various example embodiments and implementations, it should be understood that the various features, aspects, and functionality described in one or more of the individual embodiments are not limited in their applicability to the particular embodiment with which they are described, but instead may be applied, alone or in various combinations, to one or more of the other embodiments of the present application, whether or not such embodiments are described and whether or not such features are presented as being a part of a described embodiment. Thus, the breadth and scope of the present application should not be limited by any of the above-described example embodiments.

Terms and phrases used in the present application, and variations thereof, unless otherwise expressly stated, should be construed as open ended as opposed to limiting. As examples of the foregoing: the term “including” should be read as meaning “including, without limitation” or the like; the term “example” is used to provide illustrative instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future. The presence of broadening words and phrases such as “one or more,” “at least,” “but not limited to” or other like phrases in some instances shall not be read to mean that the narrower case is intended or required in instances where such broadening phrases may be absent.

Additionally, the various embodiments set forth herein are described in terms of example block diagrams, flow charts, and other illustrations. As will become apparent to one of ordinary skill in the art after reading this document, the illustrated embodiments and their various alternatives may be implemented without confinement to the illustrated examples. For example, block diagrams and their accompanying description should not be construed as mandating a particular architecture or configuration. Also, one of ordinary skill in the art will recognize, upon studying the present disclosure, how teachings in any one of the figures herein may be applied to any of the other figures herein.

What is claimed is:

1. An apparatus for impeding vertebra displacement after a spinal fusion surgery, the apparatus comprising:
an elongated tissue comprising a first end and a second end;
a first fastener attached to the first end and configured to be coupled to a first vertebra; and
a second fastener attached to the second end and configured to be coupled to a second vertebra coupled to a third vertebra via a spinal fusion instrumentation.

2. The apparatus of claim 1, wherein the elongated tissue is a cadaveric tissue harvested from a human cadaver.

3. The apparatus of claim 1, wherein the first fastener is wrapped around the first end, or the second fastener is wrapped around the second end.

4. The apparatus of claim 1, wherein the first fastener passes through the first end, or the second fastener passes through the second end.

5. The apparatus of claim 1, wherein the first fastener comprises a first strand, or the second fastener comprises a second strand.

6. The apparatus of claim 5, wherein one or more of the first and second strands comprise one or more of thread-like material, wire, and flexible synthetic material.

7. The apparatus of claim 5, wherein the first strand comprises a first loop attachable to the first vertebra, or the second strand comprises a second loop attachable to the second vertebra.

8. The apparatus of claim 1, wherein the first fastener is configured to be coupled to the first vertebra via a first anchor feature, or the second fastener is configured to be coupled to the second vertebra via a second anchor feature.

9. The apparatus of claim 8, wherein the first anchor feature comprises a first passage formed in the first vertebra, or the second anchor feature comprises a second passage formed in the second vertebra.

10. The apparatus of claim 8, wherein the first anchor feature comprises a first fixation protrusion coupled to the first vertebra, or the second anchor feature comprises a second fixation protrusion coupled to the second vertebra.

11. An apparatus, comprising:
first and second fasteners attachable to vertically adjacent vertebrae, at least one of the vertically adjacent vertebrae being coupled to an other vertebra by way of a spinal fusion; and
a tissue comprising a first end coupled to the first fastener and a second end coupled to the second fastener.

12. The apparatus of claim 11, wherein the tissue is sized in length to span a distance between the vertically adjacent vertebrae when the first fastener is attached to a first of the vertically adjacent vertebrae and the second fastener is attached to a second of the vertically adjacent vertebrae.

13. The apparatus of claim 11, wherein the tissue is selected so as to impede lateral displacement of the first of the vertically adjacent vertebrae relative to the second of the vertically adjacent vertebrae after a spinal fusion surgery.

14. The apparatus of claim 11, wherein the spinal fusion comprises one or more of a bone graft, a spinal fusion instrumentation, and a bone growth.

15. The apparatus of claim 11, wherein the first fastener comprises a first strand having a first loop attachable to a first protrusion that protrudes from a first of the vertically adjacent vertebrae, or the second fastener comprises a second strand having a second loop attachable to a second protrusion that protrudes from a second of the vertically adjacent vertebrae.

16. The apparatus of claim 11, wherein the spinal fusion comprises a spinal fusion instrumentation; and wherein one or more of the first and second fastener is attachable to the spinal fusion instrumentation.

17. A system for impeding vertebra displacement, the system comprising:
an elongated member comprising a proximal end and a distal end;
a first fastener attached to the proximal end and configured to be coupled to a first vertebra;
a second fastener attached to the distal end and configured to be coupled to a second vertebra; and
an implant coupled between the second vertebra and a third vertebra and configured to promote a fusion of the second vertebra and the third vertebra.

18. The system of claim 17, wherein the first and second vertebrae and the second and third vertebrae are vertically adjacent; and wherein the elongated member is sized so as to span a distance between the first and second vertebrae when the first fastener is coupled to the first vertebra and the second fastener is coupled to the second vertebra.

19. The system of claim 17, wherein the implant comprises a spinal fusion instrumentation; and wherein the second fastener is configured to be coupled to the second vertebra by attaching to the spinal fusion instrumentation, or the first fastener is configured to be coupled to the first vertebra by attaching to an anchor feature coupled to the first vertebra.

20. The system of claim 17, wherein the first fastener comprises a first loop attachable to the first vertebra and at least a portion of the first fastener is wrapped around the proximal end, or the second fastener comprises a second loop attachable to the second vertebra and at least a portion of the second fastener is wrapped around the distal end.
